(19) United States
(12) Patent Application Publication
(10) Pub. No.: US 2026/0013793 A1
(43) Pub. Date: Jan. 15, 2026

(54) MULTI-PARAMETER DIABETIC FOOT MONITORING SYSTEM WITH INTEGRATED COMMUNICATION PROTOCOLS

(71) Applicant: Olutosin Alabi, Bronx, NY (US)

(72) Inventor: Olutosin Alabi, Bronx, NY (US)

(21) Appl. No.: 19/266,837

(22) Filed: Jul. 11, 2025

Related U.S. Application Data
(60) Provisional application No. 63/671,195, filed on Jul. 13, 2024.

(51) Int. Cl. A61B 5/00; A61B 5/1455; G16H 40/67; G16H 50/30

(52) U.S. Cl. CPC A61B 5/6807; A61B 5/1455; G16H 40/67; G16H 50/30; A61B 2562/0215; A61B 2562/0247; A61B 2562/0271; A61B 2562/029; A61B 2562/046

(57) ABSTRACT

A multi-modal sensor system for monitoring a user includes a smart sock and smart insole configured to be worn on a foot of the use. The sock and insole each have a multi-modal sensor array integrated within their respective structures. For example, the sensor array may include a temperature sensor, a pressure sensor, a moisture sensor, and an optical sensor to measure multiple physiological parameters on the foot of the user. Processing electronics provide for early detection of inflammation, pressure ulceration risk, and circulation changes through coordinated wireless communication between the sock and insole for comprehensive diabetic foot health monitoring during continuous daily wear applications.

### BACKGROUND OF THE INVENTION

### 1. Field of the Invention

The present disclosure relates to a wearable multi-modal sensor systems for diabetic foot monitoring and, more specifically an innovative smart sock and smart insole technologies that integrates multiple sensing modalities within hybrid fabric and substrate constructions providing precision physiological measurement capabilities with comprehensive multi-parameter assessment of diabetic foot health parameters.

### 2. Description of the Related Art

Diabetic foot complications represent one of the most serious and costly consequences of diabetes mellitus, affecting millions of patients worldwide and leading to significant morbidity, mortality, and healthcare costs. Current diabetic foot monitoring approaches rely primarily on periodic clinical examinations and patient self-assessment, which are inadequate for early detection of developing complications such as pressure ulcers, infections, and circulation problems. Traditional pressure monitoring devices are typically bulky, uncomfortable, and limited to clinical settings, failing to provide the continuous, real-time monitoring necessary for effective preventive care.

Existing wearable monitoring solutions suffer from several critical limitations including poor sensor integration, limited multi-parameter capability, inadequate comfort for extended wear, insufficient accuracy for clinical applications, and lack of comprehensive foot coverage. Conventional smart textile approaches often compromise either sensing performance or wearing comfort, while traditional insole sensors are typically rigid, non-breathable, and prone to mechanical failure during normal daily activities. These existing insole systems typically employ thick, inflexible substrates that create uncomfortable pressure points and interfere with natural foot biomechanics.

Previous attempts at diabetic foot monitoring have focused on single-parameter measurement systems, such as temperature-only monitoring or pressure-only detection, which provide insufficient information for comprehensive health assessment. For example, existing smart sock technologies typically integrate basic temperature sensors but lack the multi-modal sensing capabilities necessary for detecting the complex physiological changes associated with diabetic foot complications. These systems fail to provide the redundant measurements and cross-validation needed for reliable clinical assessment.

Current smart textile solutions face significant challenges in sensor integration, particularly in achieving reliable electrical connections while maintaining fabric flexibility and washability. Conventional approaches often result in sensor systems that are visible to users, create uncomfortable textures, or fail after repeated washing cycles. Existing sock-based monitoring systems typically rely on external sensor modules that are clipped or attached to standard fabric, resulting in bulky designs that are impractical for daily wear and prone to sensor displacement during normal activities.

Traditional insole monitoring systems suffer from limited sensor coverage, typically focusing only on specific pressure points rather than providing comprehensive plantar surface monitoring. These systems often use rigid printed circuit boards that create uncomfortable walking surfaces and fail to conform to individual foot anatomies. The lack of integration between sock and insole monitoring approaches means that existing systems cannot provide the comprehensive, coordinated monitoring necessary for optimal diabetic foot care.

Existing sensor technologies also face significant limitations in achieving the precision and sensitivity required for early detection of diabetic foot complications. Temperature sensors in current systems typically lack the accuracy needed to detect subtle inflammatory changes, while pressure sensors often have insufficient resolution to identify developing ulceration risks. Moisture sensing capabilities are generally absent from existing solutions, despite the critical importance of skin hydration monitoring in diabetic foot care.

Furthermore, existing solutions lack the integrated communication capabilities necessary for coordinated multi-device monitoring and fail to provide the manufacturing scalability required for widespread clinical deployment. Current systems typically operate as standalone devices without the ability to correlate data between multiple sensing platforms or integrate with comprehensive healthcare monitoring systems. The need exists for a comprehensive, comfortable, and clinically accurate multi-parameter monitoring system that can be worn continuously during daily activities while providing real-time health assessment and early warning capabilities for diabetic foot complications through coordinated sock and insole monitoring platforms.

### BRIEF SUMMARY OF THE INVENTION

The present invention addresses the aforementioned limitations by providing a multi-modal sensor system for monitoring a user that includes both a smart sock and smart insole configured to be worn on a foot of the user, each comprising a multi-modal sensor array integrated within their respective structures. The multi-modal sensor arrays comprise at least one temperature sensor, at least one pressure sensor, at least one moisture sensor, and at least one optical sensor to measure multiple physiological parameters on the foot of the user. The system combines ultra-thin flexible substrate technologies with silver-infused nylon conductive zones in socks, bamboo fiber comfort zones, and multi-layer insole constructions, enabling continuous monitoring and early detection of diabetic foot complications through revolutionary sensor integration architectures that eliminate conventional packaging limitations while maintaining biocompatibility, antimicrobial protection, and flexibility for extended wear applications in the form of comfortable, washable socks and durable, removable insoles

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a perspective view of a smart sock according to the present invention.

FIG. 2 is a perspective view of the construction of a smart sock according to the present invention.

FIG. 3 is a cross-sectional view of the electronics for a smart sock according to the present invention.

FIG. 4 is a bottom view of a smart sock according to the present invention.

FIG. 5 is an exploded view of a smart sock according to the present invention.

### DETAILED DESCRIPTION OF THE INVENTION

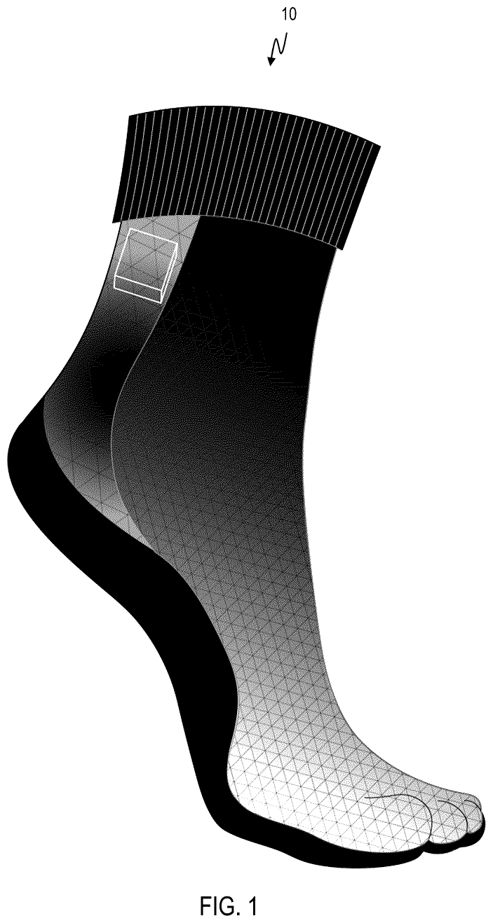

Referring to the figures, wherein like numerals refer to like parts throughout, there is seen in FIG. 1 is a perspective view of the design for a smart sock system 10, illustrating the hybrid material construction comprising silver-infused nylon zones for sensor integration, bamboo fiber blend zones for comfort, and COOLMAX polyester reinforcement zones, with an integrated processing electronics module positioned within the ankle region and configured to house processor, memory device, communication module, and power source components for real-time diabetic foot health monitoring and wireless data transmission capabilities.

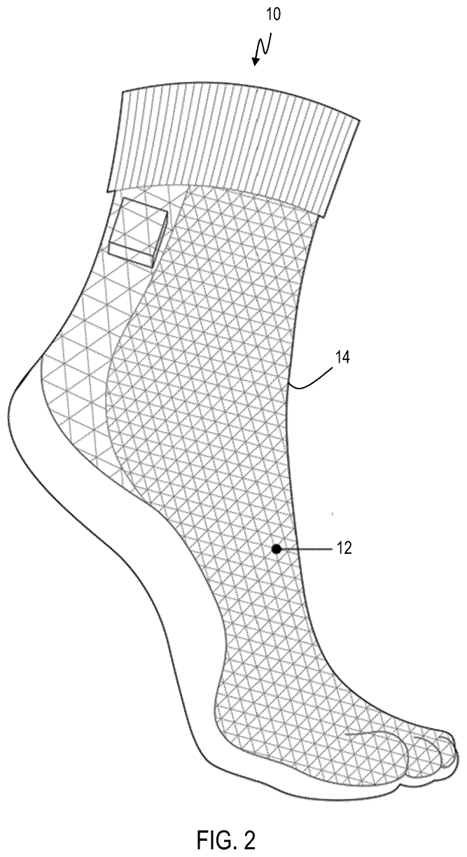

FIG. 2 is a perspective view of smart sock system 10, demonstrating the hexagonal mesh pattern fabric 12 construction throughout the main body 14 with strategically positioned multi-modal sensor integration points within the silver-infused nylon zones, illustrating the seamless integration of temperature sensors, moisture sensors, and flexible printed circuit interconnects while maintaining the natural appearance and mechanical flexibility of a conventional textile garment.

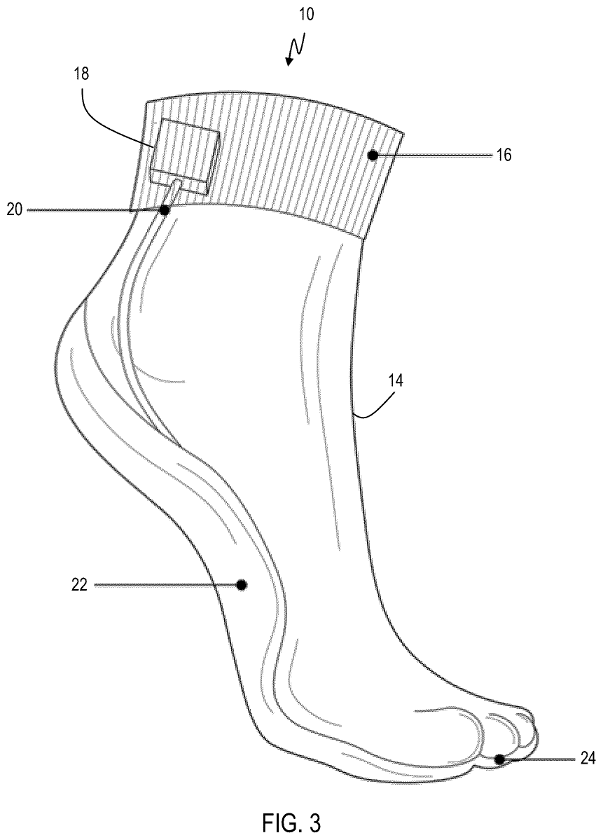

FIG. 3 is a detailed cross-sectional view of smart sock system 10 showing a fold-down cuff design 16 configured to discretely conceal the processing electronics 18 while maintaining user comfort, illustrating the sensor connectivity pathways 20 between the sock and insole components 22 via wire connections integrated between fabric layers, and demonstrating the coordinated multi-device monitoring approach with the flat insole construction featuring embedded multi-modal sensor arrays positioned at anatomically critical plantar regions including the hallux, metatarsal heads, midfoot, and heel areas for comprehensive diabetic foot complication detection. Insole 22 is a region where ulcers may sometimes occur. Toe region 24 is also susceptible to ulcers and can be provided with memory foam as well as sensors that hug the toes and provide for additional detection.

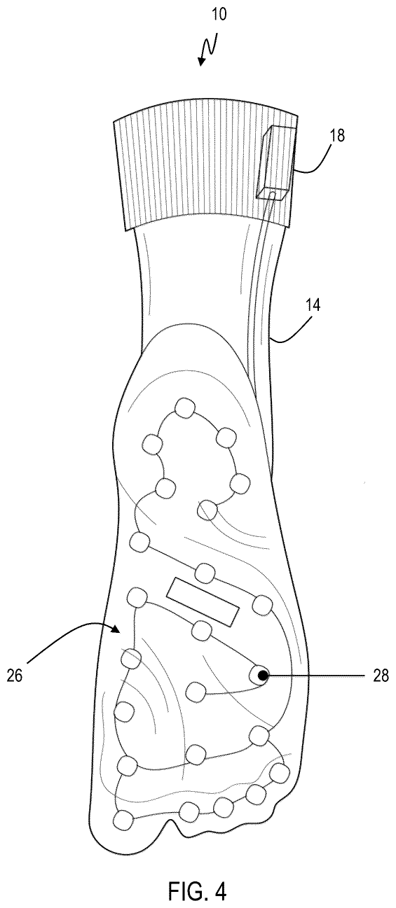

FIG. 4 is a detailed bottom view of an exemplary variation of smart sock system 10 showing the comprehensive sensor grid 26 with discrete piezoresistive sensing elements 28 strategically distributed across the entire plantar surface in an interconnected network providing high-resolution pressure mapping, illustrating how the bottom sensors connect to a centralized processing unit positioned near the ankle region via flexible printed circuit interconnects, with the insole featuring a typical flat shoe design that allows for comfortable daily wear while providing complete coverage of pressure-sensitive areas, moisture detection zones, temperature monitoring points, and optical sensor locations for vascular health assessment. Grid 26 and sensors 28 are connected to processing electronics 18 positioned near the ankle region.

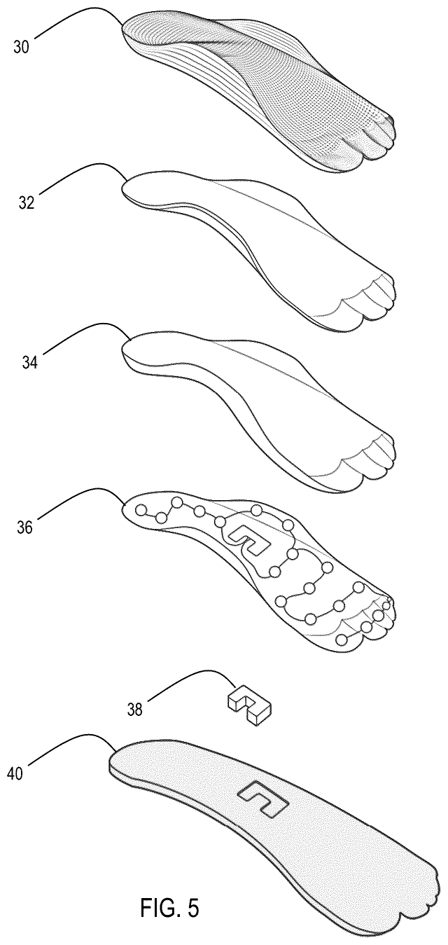

FIG. 5 is an exploded view conceptual diagram of an exemplary variation of the smart insole showing the comprehensive multi-layer construction methodology, wherein the topmost finished product layer 30 demonstrates the complete insole with anatomical arch support conforming to natural foot contours, raised lateral supports configured to increase sensor detection area for ulcer monitoring, and strategically positioned toe indentations designed to enhance early detection of developing pressure ulcers at high-risk digital locations, with the overall insole profile maintaining compatibility with standard footwear while incorporating advanced sensing capabilities.

FIG. 5 further illustrates the second layer 32 comprising the top cloth component constructed from medical-grade materials providing the user-contact interface, wherein this layer incorporates biocompatible surface treatments for extended skin contact, integrated sensor access windows positioned to allow direct sensor-to-skin proximity while maintaining protective barriers, breathable fabric construction enabling moisture management and air circulation, and antimicrobial coatings preventing bacterial growth during continuous wear applications.

FIG. 5 additionally depicts the third layer 34 showing the top cushioning component engineered to provide patient comfort while protecting embedded sensor elements, wherein this cushioning layer comprises pressure-distributing foam materials configured to prevent sensor damage during high-impact activities, strategically positioned cavities designed to house individual sensing elements while maintaining substrate flexibility, shock-absorption properties reducing mechanical stress on the underlying sensor arrays, and thermal insulation characteristics preventing external temperature interference with precision sensor measurements.

FIG. 5 demonstrates the fourth layer 36 illustrating the pressure sensing component featuring thirty-two discrete piezoresistive sensing elements 28 arranged in a high-resolution grid pattern 26 providing comprehensive plantar surface coverage, wherein each sensing element comprises conductive polymer materials screen-printed onto flexible polyimide substrates measuring less than 100 micrometers in total thickness, individual sensor cavities formed through precision laser cutting to house sensing elements while maintaining mechanical flexibility, interconnected sensor pathways enabling simultaneous multi-point pressure mapping across critical weight-bearing regions including the hallux, first through fifth metatarsal heads, midfoot arch region, and heel contact area, and calibrated sensitivity ranges optimized for detecting diabetic foot ulceration risk factors with 200 kPa threshold detection capabilities.

FIG. 5 shows the fifth layer 38 comprises a printed circuit board component strategically positioned within the arch region to minimize interference with natural gait patterns, wherein this electronic layer houses a six-axis inertial measurement unit providing accelerometer and gyroscope data for activity monitoring and gait analysis, Bluetooth 5.0 radio module enabling wireless communication with the coordinated smart sock system and external mobile devices, microprocessor with integrated memory storage for real-time data processing and temporary data buffering, power management circuitry optimized for extended battery life during continuous monitoring applications, rechargeable lithium-polymer battery pack configured for wireless charging capabilities, and miniaturized component layout utilizing advanced surface-mount technology to achieve compact form factor suitable for comfortable daily wear.

FIG. 5 illustrates the bottom foundation layer 40 constructed from durable polyurethane materials providing structural integrity and environmental protection for the complete sensor system, wherein this base layer incorporates wear-resistant properties suitable for extended daily use in various footwear configurations, environmental sealing protecting internal electronics from moisture and debris infiltration, impact-resistant construction preventing damage from normal walking and standing activities, and integrated mounting points for secure attachment of the overlying sensor and electronic components while maintaining the overall insole flexibility required for natural foot biomechanics during ambulation.

The smart sock comprises a hybrid material construction including basic textile, silver-infused nylon zones for sensor integration and bamboo fiber blend zones for comfort, while the smart insole comprises a multi-layer construction with a top layer of medical-grade polyimide, middle layer pressure distribution matrix, and bottom layer protective coating. Both the sock and insole sensor substrates measure less than 100 micrometers total thickness and comprise medical-grade polyimide construction with integrated sensor cavities, flexible printed circuit interconnects, and breathable protective coating

The multi-modal sensor array includes precision sensing elements, where the temperature sensor comprises platinum resistance temperature detectors providing ±0.1° C. accuracy, the pressure sensor comprises piezoresistive sensing elements with 200 kPa threshold detection, the moisture sensor comprises gold microelectrode arrays for electrochemical skin conductance measurement with 50 microSiemens sensitivity, and the optical sensor comprises multi-wavelength photodiodes operating at 470 nm, 525 nm, 660 nm, and 940 nm wavelengths for tissue analysis.

In some variations, the smart sock includes a hybrid material construction comprising silver-infused nylon zones positioned at sensor locations providing native electrical conductivity, superior antimicrobial properties, and EMI shielding for sensitive electronic components; bamboo fiber blend zones positioned at comfort areas providing excellent moisture-wicking, natural antibacterial properties, superior breathability, and ultra-soft texture; and COOLMAX polyester reinforcement zones at high-wear areas including heel and toc regions providing excellent durability and shape retention.

In some variations, the smart insole includes a multi-layer construction comprising a top contact layer of medical-grade silicone with integrated sensor windows providing biocompatible skin contact and protection for embedded sensors; a middle sensor substrate layer of polyimide with embedded multi-modal sensor arrays providing comprehensive foot health monitoring; a pressure distribution matrix layer with piezoresistive sensor grid providing high-resolution pressure mapping across the entire plantar surface; a bottom protective layer of durable polyurethane providing wear resistance and environmental protection; and a wireless communication module positioned within the arch region for data transmission and power management.

The invention also provides a method for making a multi-modal sensor system including forming both a smart sock with hybrid material zones and a smart insole with multi-layer construction, creating flexible polyimide sensor substrates through precision laser cutting, depositing platinum thin-film temperature sensors using DC magnetron sputtering, forming piezoresistive pressure sensors through screen printing of conductive polymer, creating gold microelectrode moisture sensors using electron beam evaporation and photolithographic patterning, bonding multi-wavelength photodiodes using automated pick-and-place equipment, and integrating sensor arrays within both the silver-infused nylon fabric zones of the sock and the polyimide substrate of the insole to create integrated multi-modal sensing platforms for continuous diabetic foot monitoring.

What is claimed is:

1. A multi-modal sensor system for monitoring a user comprising:
a smart sock configured to be worn on a foot of the user, the smart sock comprising a hybrid material construction including a plurality of silver-infused nylon zones a plurality of bamboo fiber blend zones;
a smart insole configured to be worn on the foot of the user, the smart insole comprising a multi-layer construction with a top layer of polyimide,
a middle layer pressure distribution matrix; and
a bottom layer protective coating;
wherein both the smart sock and smart insole include multi-modal sensor arrays integrated within their respective structures, each multi-modal sensor array comprising at least one temperature sensor, at least one pressure sensor, at least one moisture sensor, and at least one optical sensor configured to measure multiple physiological parameters on the foot of the user; and
wherein sensor arrays in both the smart sock and smart insole measure less than 100 micrometers total thickness.

2. The multi-modal sensor system of claim 1, wherein the temperature sensor comprises a platinum resistance temperature detector, the pressure sensor comprises piezoresistive sensing elements with 200 kPa threshold detection, the moisture sensor comprises a gold microelectrode arrays, and the optical sensor comprises a multi-wavelength photodiodes.

3. The multi-modal sensor system of claim 1, wherein the hybrid material construction further comprises:
a plurality of silver-infused nylon zones positioned at sensor locations;
a plurality of bamboo fiber blend zones; and
a plurality of reinforcement zones at high-wear areas.

4. The multi-modal sensor system of claim 1, wherein the smart insole multi-layer construction comprises:
a top contact layer of medical-grade silicone with integrated sensor windows providing biocompatible skin contact and protection for embedded sensors;
a middle sensor substrate layer of polyimide with embedded multi-modal sensor arrays providing comprehensive foot health monitoring;
a pressure distribution matrix layer with piezoresistive sensor grid providing high-resolution pressure mapping across the entire plantar surface;
a bottom protective layer of durable polyurethane providing wear resistance and environmental protection; and
a wireless communication module positioned within an arch region for data transmission and power management.

5. The multi-modal sensor system of claim 1, wherein the sensor arrays are arranged in an integrated array configurations within the silver-infused nylon zones.

6. The multi-modal sensor system of claim 1, wherein the smart sock includes integrated processing electronics positioned within an ankle region, the smart insole includes processing electronics positioned within an arch region, and wherein the integrated processing electronics include sensor signal conditioning, wireless communication capabilities, power management, and data storage.

7. The multi-modal sensor system of claim 6, wherein the integrated processing electronics are configured to provide detection of inflammation, pressure ulceration risk, and circulation changes.

8. The multi-modal sensor system of claim 1, wherein the flexible sensor substrates include integrated sensor cavities formed therein, with each cavity dimensioned to house individual sensing elements.

9. The multi-modal sensor system of claim 1, wherein the substrates include flexible printed circuit interconnects.

10. The multi-modal sensor system of claim 1, wherein the smart insole pressure sensing layer comprises thirty-two discrete piezoresistive sensing elements arranged in a high-resolution grid pattern.

11. A method for making a multi-modal sensor system. Comprising the steps of:
forming a smart sock with hybrid material construction including silver-infused nylon zones, bamboo fiber blend zones, and COOLMAX polyester reinforcement zones;
forming a smart insole with multi-layer construction including medical-grade silicone top layer, polyimide sensor substrate, pressure distribution matrix, and protective bottom layer; creating flexible polyimide sensor substrates through precision laser cutting;
depositing platinum thin-film temperature sensors using DC magnetron sputtering;
forming piezoresistive pressure sensors through screen printing of conductive polymer;
creating gold microelectrode moisture sensors using electron beam evaporation and photolithographic patterning;
bonding multi-wavelength photodiodes using automated pick-and-place equipment;
integrating sensor arrays within both the silver-infused nylon zones of the sock fabric and the polyimide substrate of the insole;
forming flexible printed circuit interconnects through additive electroplating; and
applying protective coatings through heated roll lamination to create integrated multi-modal sensing platforms for continuous diabetic foot monitoring.

12. The method of claim 11, further comprising the step of creating integrated sensor cavities within the polyimide substrate positioned within the silver-infused nylon zones, such that individual sensing elements are precisely positioned within designated cavities to maintain optimal sensor-to-skin contact while preserving substrate flexibility.

13. The method of claim 11, further comprising the step of installing miniaturized processing electronics within the ankle region of the sock and arch region of the insole, and implementing wireless communication protocols for sock-insole data coordination and smartphone connectivity.

14. The method of claim 11, wherein the step of forming the sensor arrangement includes implementing redundant sensor placement and cross-validation algorithms to ensure measurement reliability and early fault detection.

15. A smart sock system for multi-parameter monitoring, comprising:
a hybrid fabric construction having silver-infused nylon zones for sensor integration and bamboo fiber blend zones for comfort;
integrated electronics positioned within an ankle region; and
a multi-modal sensor array within the silver-infused nylon zones, wherein the sensor array comprises sensors configured to measure multiple parameters associated with diabetic foot health including temperature gradients, pressure distribution, skin moisture levels, tissue oxygenation, blood flow patterns, inflammation markers, and structural changes.

16. The smart sock system of claim 15, wherein the smart sock is designed for daily wear applications with patient comfort optimized through bamboo fiber zones and clinical accuracy optimized through silver-infused nylon sensor zones.

17. The smart sock system of claim 15, wherein the multi-modal sensor array is configured to communicate wirelessly with a coordinated smart insole system to provide comprehensive foot health assessment through redundant measurements and cross-validation.

18. The multi-modal sensor system of claim 15, wherein the protective coating is applied around a perimeter of the sensor array and throughout the substrate, thereby enclosing sensing elements within a biocompatible, breathable barrier while maintaining natural properties of the hybrid fabric construction.

19. The multi-modal sensor system of claim 15, wherein the smart insole includes a printed circuit board component housing a six-axis inertial measurement unit, Bluetooth radio module, microprocessor with integrated memory storage, power management circuitry, and rechargeable battery pack configured for wireless charging capabilities.

20. The multi-modal sensor system of claim 15, wherein the sensor arrays provide coordinated multi-device monitoring with the smart sock sensor array including temperature and moisture sensors positioned at skin contact points, and the smart insole sensor array including comprehensive pressure sensors positioned across the entire plantar surface, temperature sensors at high-risk locations, moisture sensors for detecting perspiration and skin integrity changes, and optical sensors positioned for vascular health assessment.
